(19) United States
(12) Patent Application Publication
(10) Pub. No.: US 2007/0048306 A1
(43) Pub. Date: Mar. 1, 2007

(54) Method for generating anti-variable region monoclonal antibodies

(76) Inventors: Jill M. Giles-Komar, Downingtown, PA (US); Michael A. Rycyzyn, Berwyn, PA (US); Kimberly C. Staquet, Royersford, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/512,911

(22) Filed: Aug. 30, 2006

Related U.S. Application Data
(60) Provisional application No. 60/712,619, filed on Aug. 30, 2005.

(51) Int. Cl. A61K 39/395; C12P 21/04

(52) U.S. Cl. 424/144.1; 435/70.21

(57) ABSTRACT

Methods for generating anti-variable region monoclonal antibodies in rodents are disclosed. The anti-variable region mAbs are useful as therapeutic agents, diagnostic agents or research reagents.

### CROSS REFERENCE TO RELATED APPLICATION

The present application claims priority to U.S. Provisional Patent Application Ser. No. 60/712,619 filed Aug. 30, 2005, the contents of which are completely incorporated by reference.

### FIELD OF THE INVENTION

This invention relates to the generation of anti-variable region monoclonal antibodies in a rodent.

### BACKGROUND OF THE INVENTION

The use of monoclonal antibodies (mAbs) as therapeutic reagents has become an effective approach for the treatment of various diseases. In addition, mAbs can represent a powerful tool to gain a better understanding of the immunopathogenesis of various diseases.

A standard method for the generation of mAbs consists of fusing myeloma cells with lymph node cells or splenocytes harvested from immunized BALB/c mice (Köhler and Milstein, Nature 256, 495-497 (1975); Köhler and Milstein, Eur. J. Immunol. 6, 511-519 (1976)). BALB/c mice represent the host of choice for raising mAbs since they are readily available and, when sensitized with foreign T-dependent antigens, the immune response in these mice is characterized by a polarization of T-cell derived cytokine production toward a Th2-like phenotype (reviewed in Reiner and Locksley, Ann. Rev. Immunol. 13, 151-177 (1995)). This Th2-like response is accompanied by the generation of high levels of antigen-specific IgG1 antibodies (Finkelman et al., Ann. Rev. Immunol. 8, 303-333 (1990)), which correlates with an increase in the frequency of antigen-specific B-cell clones and an increase in the number of hybrids following B-cell fusion.

Nevertheless, some antigens produce only low or undetectable antibody titers in BALB/c mice, making it difficult or impossible to generate hybrids following B-cell fusion. In addition, the generation of a mAb by the method of Kohler and Milstein is dependent upon the success of a complex biological process coupled with the success of in vitro techniques to harvest and immortalize the antigen specific B cell of interest.

In the drug development process for therapeutic mAbs, reagents are needed to understand the concentration-response relationship and to assess drug safety and efficacy in pharmacokinetic/pharmacodynamic (PK/PD) studies. The generation of anti-variable region antibodies against therapeutic mAbs has therefore become an important part of the clinical drug development process. For example, sandwich EIA is routinely utilized to achieve sensitivity and selectivity in PK/PD assays, which requires the generation of anti-variable region antibodies that bind specifically to drug in non-competing pair combinations. Thus, large panels of anti-variable region antibodies must be generated in order to increase the probability of successful pair identification.

However, the generation of anti-variable region antibodies is a labor-intensive process that typically takes 4-6 months from start of immunization to final candidate evaluation. In addition, deliberately generating an anti-variable region antibody is often not easy, and usually requires the use of adjuvant or coupling to “carrier” proteins such as keyhole limpet heamocyanin (KLH). While the use of adjuvant such as complete Freund's adjuvant or alum can boost the humoral response against foreign antigens, this procedure can denature some protein antigens. This can have a detrimental effect on the processing and presentation of key immunogenic epitopes for the generation of specific antibodies to conformational epitopes such as those is in the complementarity determining regions (CDRs).

Therefore, a need exists for methods that can rapidly generate large panels of anti-variable region antibodies in rodents such as Balb/c mice.

### SUMMARY OF THE INVENTION

One aspect of the invention is a method for generating mAbs in a rodent comprising the steps of administering a dendritic cell maturation agent to the rodent; immunizing the rodent with an antigen; and isolating antigen-specific antibodies.

Another aspect of the invention is a method for generating mAbs in a rodent comprising the steps of administering a dendritic cell maturation agent to the rodent; immunizing the rodent with an antigen; administering a B cell expansion agent to the rodent; and isolating antigen-specific antibodies.

One specific aspect of the invention is a method for generating anti-variable region mAbs in a rodent comprising the steps of administering a dendritic cell maturation agent to the rodent; immunizing the rodent with a mAb; and isolating anti-variable region mAbs.

Another specific aspect of the invention is a method for generating anti-variable region mAbs in a rodent comprising the steps of administering a dendritic cell maturation agent to the rodent; immunizing the rodent with a mAb; administering a B cell expansion agent to the rodent; and isolating anti-variable region mAbs.

A further aspect of the invention is a method for generating anti-variable region mAbs in a rodent comprising the steps of administering a dendritic cell maturation agent to the rodent; immunizing the rodent with a mAb; administering a CD40 agonist to the rodent; and isolating anti-variable region mAbs.

### DETAILED DESCRIPTION OF THE INVENTION

All publications, including but not limited to patents and patent applications, cited in this specification are herein incorporated by reference as though fully set forth.

The term “anti-Id mAb,” also referred to as “anti-idiotypic mAb,” “anti-variable region mAb,” or “peptide-specific detection mAb,” as used herein and in the claims, means any mAb specific for the target antibody variable region.

The term “dendritic cell maturation agent” as used herein and in the claims means any agent that causes the conversion of immature dendritic cells to cells that can process antigens and display antigen peptide fragments on the cell surface together with; molecules required for T-cell activation and prime naive syngeneic T-cells, known in the art as professional antigen-presenting cells (APC).

The term “in combination with” as used herein and in the claims means that the described agents can be administered to a rodent together in a mixture, concurrently as single agents or sequentially as single agents in any order.

A method for generating mAbs in a rodent comprising the steps of administering a dendritic cell expansion agent to the rodent; administering a dendritic cell maturation agent to the rodent; immunizing the rodent with an antigen; and isolating antigen-specific antibodies is described in WO 05/037314, herein incorporated by reference in its entirety.

The present invention provides methods for generating anti-variable region mAbs in rodents, such as but not limited to mice having a BALB/c background.

In one embodiment of the present invention, administration of a dendritic cell maturation agent to a rodent having a BALB/c background concurrent with immunization with a mAb antigen enhances the humoral response and elicits a rapid and increased antibody response. This method of the invention is useful in the generation of anti-variable region mAb in these animals. The antibodies generated by the method of the invention are useful as therapeutic agents, diagnostic agents or research reagents.

Maturation agents useful in the method of the invention include any cytokines that will cause the conversion of immature dendritic cells to mature professional antigen-presenting cells and potentiate T-cell activation. These agents include type I interferons, tissue necrosis factor-α, interleukin-6, prostaglandin-E2, interleukin-1α, interleukin-1β, interleukin-18, interleukin-12, interleukin-4, interleukin-23, interferon-γ, granulocyte-macrophage colony-stimulating factor or a dendritic cell-associated maturation factor agonist singly or in combination with other dendritic cell maturation agents. Dendritic cell-associated maturation factor agonists include, but are not limited to, any antibody, fragment or mimetic or small molecule agonist.

Type I interferons include interferon-α (IFN-α), interferon-β (IFN-β), IFM-δ, IFN-α1, IFN-α2, IFN-α2a, IFN-α2b, IFN-α4, IFN-αII1, IFN-αCon1, IFN-αLE, IFN-αLy or IFN-β2. Type I interferon has been shown to induce antibody production (Le Bon et al., Immunity 14, 461-470 (2001).

One of ordinary skill in the art could readily determine the amounts of dendritic cell maturation agents to administer. For example, about 10⁵ U to about 2×10⁵ U each of IFN-α and IFK-β, daily for about 3 days to about 5 days can be used to induce dendritic cell maturation.

Concurrent with or prior to administration of the dendritic cell maturation agent, the rodent is immunized with target mAb by techniques well known to those skilled in the art. After immunization of the rodent, polyclonal antibodies or clonal populations of immortalized B cells are prepared by techniques known to the skilled artisan. Anti-variable region mAbs can be identified from clonal populations by screening for binding and/or biological activity toward the target mAb of interest by using peptide display libraries or other techniques known to those skilled in the art.

Optionally, in this embodiment of the invention, mice can be further treated post-immunization with B cell expansion agent. A B cell expansion agent useful in the method of the invention is a CD40 agonist. Further examples of B cell expansion agents include but are not limited to, CD154, C3a, C3b, anti-IgM, BAFF, anti-CD80, anti-CD86 and others known in the field.

A CD40 agonist also enhances the immune response to antigens that produce low titers of antibodies. An exemplary CD40 agonist is an anti-CD40 antibody or antibody fragment such as a monoclonal anti-mouse CD40 antibody raised against a recombinant extracellular domain of mouse CD40. One of ordinary skill in the art could readily determine the amounts of anti-CD40 antibody to administer. For example, about 50 μg to about 100 μg of the anti-CD40 mAb (clone 1C10, Catalog No. MAB440, R&D Systems, Minneapolis, Minn.) administered about 3 days prior to lymphocyte harvest can be used to enhance the overall yield of antigen-reactive B lymphocytes from these mice.

In the present invention, the omission of denaturing adjuvant in the preparation of protein antigens likely allows for processing and presentation of those conformational epitopes contained in or engrafted into the CDRs of the mAb target, thereby increasing the numbers of usable mAbs despite the presence of the highly immunodominant Fc region of the mAb or mAb scaffold. This IFN based, immunostimulatory approach optimizes the humoral response for the rapid generation of anti-variable region antibodies to a target mAb, such as a therapeutic mAb candidate. It increases the overall immune response to whole molecule IgGs as compared to conventional adjuvant. Moreover, it overcomes the dominant immune response to the Fc portion of IgG, providing a significant advantage over the use of conventional adjuvant.

Having generally described the invention, the same will be more readily understood by reference to the following example, which is provided by way of illustration and is not intended as limiting.

### EXAMPLE

### Generation of Anti-Variable Region mAbs in BALB/c Mice

BALB/c mice (8 to 12 weeks old) were purchased from The Jackson Laboratory (Bar Harbor, Me.). Recombinant murine IFNα (Catalog No. PMC4016) and IFNβ (Catalog No. PMC4024) were purchased from Biosource (Camarillo, Calif.).

Anti-variable region mAbs were generated in two BALB/c mouse treatment groups against a therapeutic mAb candidate (target mAb). In group 1, mice were immunized on day 1 with the whole IgG target mAb in combination with IFNα and IFNβ (IFNα/β). Mice received two more injections of IFNα/β on days 2 and 3. A total amount of 10⁵ U of IFNα and 10⁵ U of IFNβ were injected into each mouse over the 3-day period. On day 14, each mouse received a boost dose of the target mAb in combination with 100 μg anti-murine CD40 mAb (clone 1C10, Catalog No. MAB440, R&D Systems) through subcutaneous injection. On day 18, the mice were sacrificed and lymphocytes were harvested. In comparison, mice in group 2 were immunized with the whole IgG target mAb emulsified in Freund's adjuvant and given at least three biweekly boost injections. Three days prior to lymphocyte harvest, each mouse received 100 μg anti-murine CD40 mAb. Mice in group 2 were sacrificed and lymphocytes were harvested at various points (from day 105 to day 176).

Serum titer responses to the whole IgG target mAb were measured by sandwich ELISA and the results demonstrated a comparable immune response to the whole IgG target mAb between mice immunized via IFN and via Freund's adjuvant (data not shown).

The harvested lymphocytes were fused with murine myeloma cells and hybrids were generated by standard hybridoma techniques. The fusions were screened by ELISA to assess the number of reactive hybrids. Positive hybrids were subsequently cross-screened against several related human IgG to assess variable region specificity and these data were compared between the immunization groups. It was shown the use of IFNs significantly increased the number both of whole molecule reactive hybrids and more specifically, of anti-variable region mAbs generated in a shorter timeframe. Seventy-two variable region-specific mAbs were generated to the target mAb from three IFN fusions on day 18. In contrast, 14 variable region-specific mAbs were generated from six conventional adjuvant fusions on days 105-176.

It will be clear that the invention can be practiced otherwise than as particularly described in the foregoing description and examples. Numerous modifications and variations of the present invention are possible in light of the above teachings and, therefore, are within the scope of the appended claims.

1. A method for generating anti-variable region monoclonal antibodies in a rodent comprising the steps of:
a) administering a dendritic cell maturation agent to the rodent;
b) immunizing the rodent with a target mAb;
c) isolating anti-variable region monoclonal antibodies.

2. A method for generating anti-variable region monoclonal antibodies in a rodent comprising the steps of:
a) administering a dendritic cell maturation agent to the rodent;
b) immunizing the rodent with a target mAb;
c) administering a B cell expansion agent to the rodent;
d) isolating anti-variable region monoclonal antibodies.

3. The method of claim 1 or 2 wherein the dendritic cell maturation agent is a type I interferon, tissue necrosis factor-α, interleukin-6, prostaglandin-E2, interleukin-1α, interleukin-1β, interleukin-18, interleukin-12, interleukin-4, interleukin-23, interferon-γ, granulocyte-macrophage colony-stimulating factor or dendritic cell associated maturation factor agonist monoclonal antibody.

4. The method of claim 3 wherein the dendritic cell maturation agent is adminstered singly or in combination with another dendritic cell maturation agent.

5. The method of claim 3 wherein the type I interferon is at least one of interferon-α (IFN-α), interferon-β (IFN-β), IFN-δ, IFN-α1, IFN-α2, IFN-α2a, IFN-α2b, IFN-α4, IFN-αII1, IFN-αCon1, IFN-αLE, IFN-αLy or IFN-β2.

6. The method of claim 5 wherein the type I interferon is a combination of IFN-α and IFN-β.

7. The method of claim 2 wherein the B cell expansion agent is at least one of CD40 agonist, CD154, C3a, C3b, anti-IgM, BAFF, anti-CD80, or anti-CD86.

8. The method of claim 7 wherein the B cell expansion agent is a CD40 agonist.

9. The method of claim 8 wherein the CD40 agonist is an anti-CD40 antibody.

10. The method of claim 9 wherein the anti-CD40 antibody is administered in an amount of about 50 μg to about 100 μg per dose.

11. The method of claim 1 or 2 wherein the rodent is a mouse.

12. The method of claim 11 wherein the mouse is a BALB/c mouse.

13. The method of claim 1 or 2 wherein the rodent is a rat.
